(19) United States
(12) Patent Application Publication
(10) Pub. No.: US 2020/0367777 A1
(43) Pub. Date: Nov. 26, 2020

(54) ELECTRODE STRUCTURE FOR ELECTROCARDIOGRAM (ECG) WAVEFORM MEASUREMENT

(71) Applicant: MSL INC., Daejeon (KR)

(72) Inventors: Hee Gon Kim, Daejeon (KR); Hyun Ju Oh, Daejeon (KR); Sang Dong Choi, Daejeon (KR); Young Hwan Kang, Daejeon (KR)

(21) Appl. No.: 16/636,159

(22) PCT Filed: Sep. 12, 2018
(86) PCT No.: PCT/KR2018/010684

§ 371 Date: Feb. 3, 2020

(30) Foreign Application Priority Data
Sep. 18, 2017 (KR) 10-2017-0119453

(51) Int. Cl. A61B 5/0408; A61B 5/0428

(52) U.S. Cl. CPC A61B 5/0408; A61B 2562/0215; A61B 5/0428

(57) ABSTRACT

An electrode for electrocardiogram (ECG) waveform measurement of the present disclosure is proposed. The present disclosure provides an electrode device capable of accurately measuring and monitoring an electrocardiogram of a person by maintaining a uniform amount of electric charge even when a contact area of the electrode changes due to vigorous physical activity such as walking or running, or moisture permeation due to ambient conditions or sweat released during exercise.

### CROSS-REFERENCE TO RELATED APPLICATIONS

This application is the U.S. national phase of the International Patent Application No. PCT/KR2018/010684 filed Sep. 12, 2018, which claims the benefit of Korean Patent Application No. 10-2017-0119453 filed Sep. 18, 2017, the entire content of which is incorporated herein by reference.

### FIELD

The present disclosure relates to an electrode device capable of reducing motion noise occurring in the skin of a person when measuring an electrocardiogram during physical activity, and capable of more reliably detecting biosignals without digital signal processing.

### BACKGROUND

Recently, myocardial infarctions, arrhythmias, or the like occur without warning to patients with heart disease as well as healthy general people, and thus the occurrences of fatal threats are increasing. In order to prevent such problems and lead a healthy life, many technologies which converge IT technology for measuring biosignals are being developed to identify abnormal signs of human body through continuous biosignal monitoring and recording and to take action in case of the abnormalities.

The human body is a kind of conductor in which an electrical field is generated, and the electrical field is generated in the human body due to an action potential generated by electrical excitation and stabilization of cells. The electrical field may be interpreted as a result value for everyday physical phenomena so as to measure the abnormality, movement, and activity of the living body.

In general, a biosignal measurement using this principle may measure an electrocardiogram (ECG), body temperature, pulse rate, blood pressure, and body changes. Also, a bioelectrode is used to detect a change of the biosignals.

In order to always wear the bioelectrode for measuring the biosignals in daily life, skin trouble should be reduced by decreasing the area of an electrode in contact with the skin. Also, the motion noise is required to be reduced to accurately measure the biosignals even during exercise.

The most representative biometric information is the electrocardiogram (ECG), which interprets the electrical activity of the heart. The ECG is a graph that shows a time-varying curve by measuring minute action potential difference generated in a myocardium when the heart beats with an electrode attached to the surface of the living body.

Since the devices for measuring the biosignals detect electrical signals on the surface of the skin, the devices should be able to detect stable signals even when users move.

Currently, an Ag/AgCl gel electrode is generally used for the electrode used by directly attached to the skin for the electrocardiogram (ECG) measurement. However, such a conventional electrode has a large resistance value and is in direct contact with the skin. Accordingly, the area of contact with the electrode is changed depending on a body movement or a measurement position when measuring the amount of electric charge generated by a cardiac muscle movement. Therefore, there is a problem in that the signals measured are distorted due to a changed amount of electric charge measured.

In addition, the Ag/AgCl electrode uses a gel type electrolyte between the skin and the electrode to improve the contact with the skin, but the electrolyte has a problem wherein skin trouble may be caused and solidification at prolonged exposure to the human body may occur.

### SUMMARY

An objective of the present disclosure has been proposed to solve the above problems in the related art and to provide an electrode device capable of accurately measuring and monitoring an electrocardiogram of a person by maintaining a uniform amount of electric charge even when a contact area of the electrode changes due to vigorous physical activity such as walking or running, or moisture permeation due to ambient conditions or sweat released during exercise.

An electrode device according to an embodiment for real-time ECG signal acquisition comprises: an upper electrode layer 110 made of a conductive material to transmit signals; a transmission line 111 coupled to the upper electrode layer 110 to transmit measured signals to a receiving device; a dielectric layer 130 made of a biocompatible material coated on one side of the upper electrode layer 110 to be in direct contact with the skin; a lower electrode layer 120 offsetting frictional electricity by clothing and providing a reference point for the signals; a ground line 121 coupled to the lower electrode layer 120 and connected to a ground part for a reference potential; and an insulation layer 140 insulating between the upper electrode and the lower electrode. Also, the electrode device according to an embodiment comprises a sensing part wherein the sensing part is coated with a biocompatible nano inorganic material (Nano Inorganic Materials) on the upper electrode layer of the metal material. In particular, the sensing part is coated with a silica containing an alkali metal (SiO₂, hereinafter, the silica is collectively referred to as a silica containing alkali metals). The sensing part plays a role of gradually increasing or decreasing the voltage change on the basis of a relation that the higher dielectric constant is, the longer relaxation time is. To this end, the sensing part utilizes a parallel plate capacitor in which the higher dielectric constant and the thinner dielectric material are, the larger capacitance is. The sensing part plays the role on the basis of the formula for calculating the capacitance of the parallel plate capacitor C=εA/d, where C: capacitance, ε: dielectric constant, A: dielectric area in contact with a metal plate, and d: dielectric thickness. The sensing part also plays the role on the basis of the interaction formula of dielectric relaxation time τ=RC, where τ: average time electric charge is present, R: resistance, and C: capacitance. Therefore, the sensing part enables stable signal transmission even though the contact with the skin is not perfect due to physical activity, and comprises a function of eliminating fast-moving high-frequency noise.

Also, the electrode device according to an embodiment comprises: a transmission line attached to the upper electrode layer for transferring sensed information; a lower electrode layer which stabilizes circuit operation and provides a reference voltage so that the signal output from the sensing part is not distorted by the frictional electricity or the surrounding environment; a grounding part attached to the lower electrode layer, the grounding part configured to maintain a reference voltage; and an insulating layer supporting and insulating the upper electrode layer and the lower electrode layer.

In addition, the sensing part enables to measure the signals by using the fiber of the garment as a dielectric even when the electrode is not directly in contact with the skin.

The electrode device for ECG waveform measurement of the present disclosure spreads nano silica (SiO₂), which is biocompatible, on an upper electrode layer made of metal to accumulate and sequentially transfer electric charge generated between the skin and the upper electrode layer by cardiac muscle movement. Accordingly, stable signals output without signal distortion caused by a change in the amount of electric charge that may be generated by physical activity such as walking, running, and sitting of a person to whom the electrode device is attached. Thus, there is an effect that an accurate measurement of the electrocardiogram is possible without being affected by the surrounding environment.

### BRIEF DESCRIPTION OF THE DRAWINGS

Various embodiments are illustrated in the drawings, in which:

FIG. 1 is a perspective view illustrating an electrode according to the present disclosure.

FIG. 2 is a block diagram illustrating the electrode according to the present disclosure.

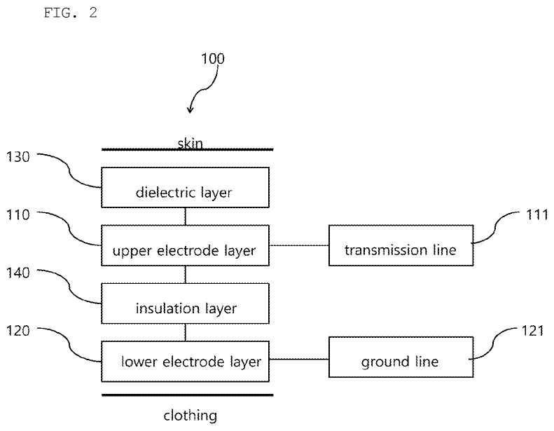

FIG. 3 is an ideal ECG waveform.

FIG. 4 is an ECG waveform measured using the electrode according to the present disclosure in the absence of physical activity.

FIG. 5 is an ECG waveform measured using the electrode according to the disclosure during physical activity.

### DETAILED DESCRIPTION

Hereinafter, an embodiment of the present disclosure is described in detail with reference to the accompanying drawings.

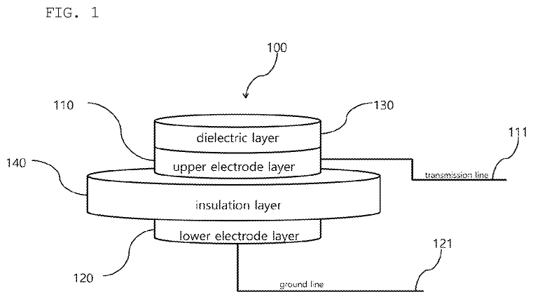

FIG. 1 is a perspective view illustrating an electrode device according to the present disclosure.

The electrode device 100 of the present disclosure comprises: an upper electrode layer 110 providing an electrode of a sensing part by using a conductive material to transmit signals; a transmission line 111 coupled to the upper electrode layer 110 to transmit sensed signals to a receiving device; a dielectric layer 130 accumulating electric charge, the dielectric layer made of a biocompatible material coated on one side of the upper electrode layer 110 to be in direct contact with the skin; a lower electrode layer 120 providing a ground part electrode made of a conductive material for offsetting frictional electricity by clothing or textiles, and providing a reference point for the signals; a ground line 121 coupled to the lower electrode layer 120 and connected to a ground part for a reference potential; and an insulation layer 140 serving as an insulation between the upper electrode and the lower electrode.

The upper electrode layer 110 may be made of a conductive material such as circular copper (Cu) of 1 cm to 10 cm in diameter with a thickness of several μm (micrometer) or less. The thickness of the dielectric layer 130 coated with a thin film on the upper electrode layer 110 shows a better effect as the thickness is reduced at the level of nm (nanometer) unit. The thickness of the dielectric layer presents best results when being coated more than 20 nm (nanometer) and less than 2 μm (micrometer). The upper electrode layer 110 is not limited to a circular shape and may be provided in various shapes.

Here, the upper electrode layer 110 may be made of conductive metals comprising gold (Au), silver (Ag), platinum (Pt), copper (Cu), and stainless steel and a conductive material comprising conductive rubber (comprising fibers).

The dielectric layer 130 made of silica (SiO₂), which is a nano inorganic material, is coupled to one surface of the upper electrode layer 110 to accumulate the electric charge so that the ECG output signals are not distorted even when the human body moves.

Practically, the dielectric layer 130 uses biocompatible dielectric materials which do not cause irritation upon skin contact, such as skin-friendly silica (SiO₂) containing alkali metals, and which are nano inorganic materials.

The transmission line 111 is coupled to the upper electrode layer 110 to transmit information sensed from the electrode layer to the receiving device.

Practically, the transmission line 111 uses a coaxial cable in order to reduce noise caused by external electrical interference.

The insulation layer 140 is fixed to the upper electrode layer 110 and the lower electrode layer 120 to enable insulation therebetween.

The insulation layer 140 may be made of an insulating material such as polyimide as a polymer material.

The lower electrode layer 120 removes the noise caused by the frictional electricity in a way that the frictional electricity generated by clothing or textiles, that is, the effect of the external environment, is eliminated by connecting to the ground electrode of the measurement system through the ground line 121.

The lower electrode layer 120 may be made of the same material, the same thickness, the same size, and the same shape as the upper electrode layer 110. However, different conductive materials, thicknesses, sizes, and shapes may be used to make up the electrode layers in one electrode device.

Practically, the ground line 121 uses a metal cable having a low resistance.

The electrode device 100 of the present disclosure is compatible with the existing receiver by adding and connecting a signal conversion device to an existing electric charge transfer type receiver.

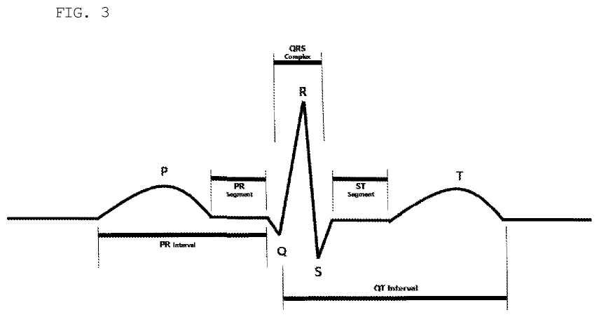

FIG. 3 is a diagram illustrating an electrical current of the heart muscle with an ideal ECG waveform. Typical electrocardiogram graphs show P waves representing atrial depolarization, QRS waves representing ventricular depolarization, and T waves representing ventricular repolarization. The time interval or distance interval of each waveform represents the conduction time according to the electrogenesis of each muscle, and the PR interval is formed within 0.12 to 0.2 seconds in normal cases, and becomes the atrioventricular nodule conduction time. The interval of QRS wave occurs within 0.06 to 0.1 seconds in normal cases, and is the time when ventricular depolarization occurs. QT interval occurs within about 0.42 to 0.43 seconds in normal cases, as an electrical systole of the electrocardiogram.

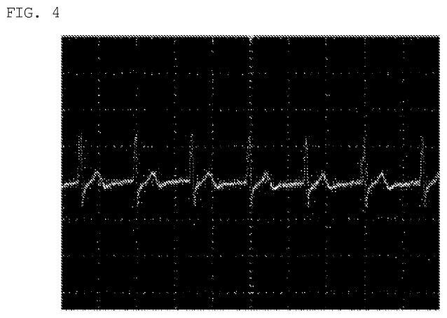

FIG. 4 is an electrocardiogram waveform measured by a detailed embodiment according to the present disclosure and measured during a stationary state or inactivity.

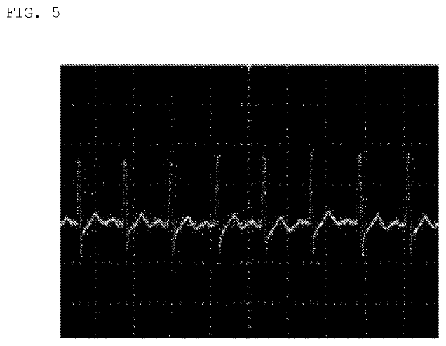

FIG. 5 is an electrocardiogram waveform measured by the detailed embodiment of the present disclosure in an active environment such as walking, jumping, or running.

1. An electrode for electrocardiogram waveform measurement,
wherein the electrode is a biosignal measuring electrode and comprises:
an upper electrode layer made of a conductive material to transmit signals;
a transmission line made of a coaxial cable, and coupled to the upper electrode layer so as to transmit measured signals to a receiving device;
a dielectric layer made of a biocompatible nano inorganic material coated on a side of the upper electrode layer to be in direct contact with skin;
a lower electrode layer grounded to offset frictional electricity generated by clothing;
a ground line coupled to the lower electrode layer and connected to a ground part; and
an insulation layer insulating between the upper electrode layer and the lower electrode layer.

2. The electrode for electrocardiogram waveform measurement of claim 1, wherein the conductive material is made of gold (Au), silver (Ag), platinum (Pt), copper (Cu), and stainless steel to transmit the signals.

3. The electrode for electrocardiogram waveform measurement of claim 1, wherein the upper electrode layer is provided in a circular shape of 1 cm to 10 cm in diameter.

4. The electrode for electrocardiogram waveform measurement of claim 1, wherein the dielectric layer is made of a biocompatible dielectric material.

5. The electrode for electrocardiogram waveform measurement of claim 1, wherein the dielectric layer has a thickness of 20 nm (nanometer) or more and 2 μm (micrometer) or less.

6. The electrode for electrocardiogram waveform measurement of claim 1, wherein the nano inorganic material is made of silica (SiO₂) containing alkali metal.

7. The electrode for electrocardiogram waveform measurement of claim 1, wherein the biosignal measuring electrode is capable of measuring the signals during physical activity.
